(19) United States
(12) Patent Application Publication
(10) Pub. No.: US 2007/0213618 A1
(43) Pub. Date: Sep. 13, 2007

(54) SCANNING FIBER-OPTIC NONLINEAR OPTICAL IMAGING AND SPECTROSCOPY ENDOSCOPE

(75) Inventors: Xingde Li, Seattle, WA (US); Daniel J. MacDonald, Seattle, WA (US); Mon Thiri Myaing, Ann Arbor, WA (US)

Correspondence Address: LAW OFFICES OF RONALD M ANDERSON, 600 108TH AVE, NE, SUITE 507, BELLEVUE, WA, 98004, US

(73) Assignee: University of Washington, Seattle, WA (US)

(21) Appl. No.: 11/623,974

(22) Filed: Jan. 17, 2007

Related U.S. Application Data
(60) Provisional application No. 60/759,405, filed on Jan. 17, 2006.

(51) Int. Cl. A61B 5/00; A61B 1/06

(52) U.S. Cl. 600/476; 600/182

(57) ABSTRACT

A miniature, flexible, fiber-optic scanning endoscope for nonlinear optical imaging and spectroscopy. The endoscope uses a tubular piezoelectric actuator for activating a cantilevered optical fiber from which pulsed light produced by a laser source exits and is directed to a target region through a micro-lens. The actuator is activated by two modulated signals that achieve two-dimensional beam scanning in a desired scan pattern. A double-clad optical fiber is employed for delivery of the excitation pulsed light and collection of emitted light received from the target region. The pulsed light travels through a core of the double-clad optical fiber, and the emitted light from the target region is directed into the core and inner cladding of the optical fiber and conveyed to a proximal end, for detection and processing. The emitted light can include multiphoton fluorescence, second harmonic generation light, and spectroscopic information, for use in imaging.

### RELATED APPLICATIONS

This application is based on a prior copending provisional application, Ser. No. 60/759,405, filed on Jan. 17, 2006, the benefit of the filing date of which is hereby claimed under 35 U.S.C. § 119(e).

### GOVERNMENT RIGHTS

This invention was funded at least in part with a grant (No. BES-0348720) from the National Science Foundation, a grant (No. 5R21EB0032840-02) from the National Institutes of Health, and the U.S. government may have certain rights in this invention.

### BACKGROUND

When tissue at a target site is illuminated with short pulses of light (with a pulse duration ranging from picoseconds to femtoseconds) emitted by a laser source, fluorophore molecules (either intrinsic or extrinsic) at the target site absorb photons of the light. The quantity of intrinsic fluorophore molecules such as NADH or FAD can indicate local metabolic activity, which can be used for detecting diseases. (NADH is Nicotinamide Adenine Dinucleotide (NAD) plus Hydrogen, i.e., the reduced form of NAD, while FAD is Flavin Adenine Dinucleotide.) Extrinsic fluorophore molecules (such as fluorescence labeled antibodies and ligands) are typically introduced into the tissue and can preferentially bind to specific cells or cell organelles of specific types of tissue, such as abnormal or cancerous tissue. The absorption of photons by the fluorophore molecules at the target site pumps electrons comprising the molecules from their normal ground state to higher excited energy levels. There is some radioactive decay, and then, when the electrons return to the ground energy state, they emit photons comprising a characteristic fluorescence light having a substantially lower energy, and therefore, longer wavelength than the exciting photon that was absorbed by the electron. Because the wavelengths of the exciting light pulses and the emitted fluorescence light from the fluorophore molecules are substantially different, they can readily be distinguished.

However, for most intrinsic fluorescence molecules to generate fluorescence, absorption of a single exciting photon from a laser pulse source requires the exciting light to be in the visible range. Unfortunately, visible light has a limited penetration depth in tissue. As an alternative to single photon excitation, the fluorescence molecules can be excited to produce fluorescence light as a result of the simultaneous absorption of two or more excitation photons of a longer wavelength (often with a deeper penetration depth in tissue, compared to shorter wavelengths), such as photons of near infrared light, that pump the electrons to the higher energy levels. The resultant emitted light is thus called multiphoton fluorescence (MPF) light. The lowest order multiphoton fluorescence process involves simultaneous absorption of two excitation photons. In this case, the process is called two-photon fluorescence (TPF). The efficiency of MPF is inversely proportional to the temporal pulse width of the excitation light. In general, the shorter the pulse width is, the higher will be the MPF efficiency.

Detection of the fluorescence light emitted from fluorophore molecules can thus be used for forming images of the target site showing the specific location of the tissue that includes the fluorophore molecules. Medical personnel can review the images to detect the presence and location of that specific tissue by thus imaging the MPF light.

A microscope is often used to image the fluorescence light emitted from fluorophore molecules in tissue of a target region. Further, for evaluating the condition of tissue at a target site within a patient's body, a fiber optic endoscope can be introduced into a patient's body and advanced to the site; the signal produced by the endoscope is then used for imaging the fluorescence light on a display. MPF imaging is now recognized as a powerful modality with unique characteristics that can provide high-resolution biochemical or molecular information complementary to the information provided by other biological imaging technologies.

The advantages of MPF imaging, particularly if not limited to ex vivo microscopy studies of tissue samples, include an intrinsic optical sectioning ability (due to a nonlinear multiphoton excitation process), deeper penetration depth into tissue (for example, as a result of using near infrared excitation light), and reduced photo-bleaching and photo-toxicity in the out-of-focus regions (due to the confinement of fluorescence excitation to the focal region). Recently, extensive research efforts have focused on developing miniature probes for MPF endoscopic imaging. Major challenges for such devices are beam scanning, efficient excitation light delivery, MPF signal collection, and probe miniaturization.

A nonlinear process similar to TPF can also occur in materials with a non-centrosymmetric molecular organization (such as a muscle fiber bundle, cartilage, or a well-organized collagen network in other types of tissue). In this case, two excitation photons are absorbed and excite the electron of the non-centrosymmetric molecule to a virtual higher energy state. Then, the excited electron relaxes to its ground state, resulting in a photon emission. The emitted photon has an energy that is equal to the sum of the energy of the two excitation photons (or twice as much as a single excitation photon). This process is called second harmonic generation (SHG). The non-centrosymmetric molecule that produces SHG photons or light is referred to herein as an “SHG molecule.” The SHG signal produced by detecting SHG light emitted from SHG molecules can reveal the integrity of the local tissue organization, which in turn, can be used for disease detection (such as the detection of cancerous tissue). Thus, the evaluation of tissue at a site based upon SHG light as well as upon the MPF emitted from the site when excited by incident excitation light can provide more complete information applicable to diagnostic functions.

### SUMMARY

This following discussion is directed to a scanning optical fiber endoscope for real-time imaging, e.g., for producing MPF and SHG images, as well as collecting spectroscopic information, which addresses the challenges mentioned above. Two-dimensional beam scanning is realized by resonantly scanning a fiber-optic cantilever with a tubular piezoelectric actuator. A double-clad optical fiber is used for delivery of excitation light and collection of emitted light from the internal target region. Detection electronics and the majority of the optical components including a dispersion compensator and dichroic mirror are placed at the input (or proximal) end of the flexible endoscope. The relatively few components required at the distal end include a small piezoelectric actuator configured to drive a cantilevered optical fiber to scan the target region, and a focusing lens, simplifying the alignment of these components and making the endoscope flexible and very compact.

More specifically, a system is described for capturing nonlinear optical images of a target region within a patient's body and providing other output information, including spectroscopic images. An exemplary embodiment of the system includes a light source that produces a pulsed light. An optical fiber having a core covered by a plurality of claddings extends between a proximal end and a distal end. The core is configured to couple at the proximal end of the optical fiber to the light source that is producing the pulsed light and conveys the pulsed light to the distal end of the optical fiber. A cantilevered optical fiber that includes a core within a plurality of claddings is coupled to the distal end of the optical fiber to receive the pulsed light, so that the pulsed light is conveyed through the core of the cantilevered optical fiber and exits from a free end of the cantilevered optical fiber. An actuator is included for driving the cantilevered optical fiber to move relative to one or more axes, so that the pulsed light exiting from the free end scans in a desired scanning pattern. The pulsed light exiting from the free end of the cantilevered optical fiber is focused by a lens toward a target region within a patient's body. The pulsed light excites molecules at the target region to emit light in response to the pulsed light, and the lens also focuses emitted light received from the target region back into the core and into an inner cladding of the cantilevered optical fiber. This emitted light is conveyed through the cantilevered optical fiber and through the core and an inner cladding of the optical fiber that is coupled thereto toward the proximal end of the optical fiber. At the proximal end of the optical fiber, a splitter is provided to separate the emitted light conveyed through the optical fiber along a detection path, from the pulsed light produced by the light source that is conveyed into the core of the optical fiber. An optical filter disposed in the detection path passes the emitted light, but rejects light having other wavelengths, such as the pulsed light and any background light that may be traveling along the detection path.

In one exemplary embodiment, a photodetector disposed in the detection path responds to the fluorescence light and produces a corresponding electrical output signal, while in another embodiment, the photodetector comprises a spectrometer and imaging device that produces an output signal indicative of spectroscopic information. The electrical output signal is processed by a processor for use determining characteristics of the internal region, e.g., for creating an image of the target region based upon the fluorescence light, or producing a spectrogram indicative of the intensity of different wavelengths in the MPF emission from the internal region.

Yet another exemplary embodiment includes a photodetector that is responsive to SHG, producing an output signal that is processed to produce SHG images of the internal region.

The splitter can include a dichroic mirror that transmits light of a first waveband (or range of wavelengths), while reflecting light of a second waveband that is substantially different than the first waveband. In this case, the pulsed light has a waveband that is substantially equal to one of the first and the second wavebands, while the emitted light has a waveband that is substantially equal to the other of the first and second waveband. Thus, the dichroic mirror can either transmit the pulsed light and reflect the emitted light, or transmit the emitted light and reflect the pulsed light.

In one exemplary embodiment, the actuator drives the cantilevered optical fiber to move in the desired scanning pattern defined relative to two generally orthogonal axes. The actuator is energized by a drive signal modulated with a voltage waveform selected from either a triangular waveform or a sinusoidal waveform (or modified versions of these basic drive waveforms). While other types of actuators can be employed, in this exemplary embodiment, the actuator comprises a tubular piezoelectric actuator.

Also included in the exemplary embodiment is a pulse dispersion manager that is disposed in a path between the light source of the pulsed light and proximal end of the optical fiber. The pulse dispersion manager negatively pre-chirps pulses of the pulsed light to compensate for a pulse broadening that is caused by a positive dispersion of the pulsed light within the core of the optical fiber. One exemplary embodiment of the pulse dispersion manager comprises a pulse stretcher that includes a grating, a lens, a folding mirror, and a reflective surface. In another exemplary embodiment, a photonic bandgap fiber (PBF) is employed as the pulse dispersion manager. In this exemplary embodiment of the pulse dispersion manager, a coupling lens is used at the input end (to couple short pulses into the PBF) and at the output end (to facilitate the coupling of short pulses into the double-clad optical fiber). The introduction of a PBF for pulse prechirping significantly reduces the overall system size and substantially reduces the excitation power loss that generally occurs in a pulse stretcher that has the grating and lens, thus allowing the use of a more compact and lower-cost short pulse laser source.

A lens can be included for coupling the pulsed light into the core at the proximal end of the optical fiber. The lens that focuses pulsed light exiting from the free end of the cantilevered optical fiber can comprise a micro lens such as a gradient index (GRIN) lens, or an achromatic micro-compound lens.

The light source that produces the pulsed light in this exemplary embodiment comprises a laser that produces pulses with a width on the order of about several femtoseconds to several tens of picoseconds.

Another aspect of this technology is directed to a method for producing light emission from a target region in a patient's body. The method includes the steps of introducing pulsed light into a proximal end of an optical fiber having a core and a plurality of cladding layers, so that the pulsed light is conveyed by the core to a distal end of the optical fiber. The distal end of the optical fiber is configured to be advanced to a position proximate to the target region. A scanning device disposed at the distal end of the optical fiber where the scanning device receives the pulsed light is activated to move so that the pulsed light scans the target region in a desired scanning pattern. The pulsed light from the scanning device is focused onto the target region causing molecules at the target region to emit light. Emitted light received from the target region is focused into the scanning device and is conveyed through the core and an inner cladding layer of the optical fiber, to the proximal end of the optical fiber. The emitted light exiting the optical fiber is directed so that the emitted light is incident on a photodetector. This photodetector produces a signal indicative of an intensity of the emitted light as the target region is scanned in the desired scanning pattern. The signal is processed to determine a characteristic of the target region, e.g., to produce an MPF image of the target region, or to produce an SHG image of the target region, or to produce a spectroscopic image of the target region that is wavelength dependent on the emitted light. Thus, the steps of the method are generally consistent with the functions of the system discussed above.

This Summary has been provided to introduce a few concepts in a simplified form that are further described in detail below in the Description. However, this Summary is not intended to identify key or essential features of the claimed subject matter, nor is it intended to be used as an aid in determining the scope of the claimed subject matter.

### DRAWINGS

Various aspects and attendant advantages of one or more exemplary embodiments and modifications thereto will become more readily appreciated as the same becomes better understood by reference to the following detailed description, when taken in conjunction with the accompanying drawings, wherein:

FIG. 1A is a schematic of the miniature beam scanning head that includes a cantilevered optical fiber having a core within a plurality of claddings that is driven to vibrate in a desired scanning pattern by an actuator;

FIG. 1B illustrates the shape of exemplary amplitude-modulated drive waveforms for the x and y electrodes of the beam scanning head;

FIG. 1C illustrates an exemplary resultant spiral scan pattern;

FIG. 2A illustrates a cross-sectional structure of an exemplary double-clad optical fiber, wherein excitation light propagates in the core to scan a sample or target region, while emitted light from a sample or target region is collected and conveyed through the core and the inner cladding;

FIG. 2B is a schematic illustration of an exemplary fiber-optic scanning endoscope imaging system, wherein the emitted light from the sample or target region is collected by a micro-lens and the cantilevered optical fiber, conveyed through a double-clad optical fiber, and then directed towards a photodetector, which produces an output signal in response to the emitted light, for processing to determine a characteristic of the sample or target region;

FIG. 2C is a schematic diagram illustrating one exemplary embodiment of a pulse dispersion manager that includes a grating, lens, and reflector, for pulse stretching to compensate for pulse dispersion;

FIG. 2D is a schematic diagram illustrating another exemplary embodiment of a pulse dispersion manager comprising a photonic bandgap filter of a length and appropriate characteristic selected to compensate for pulse dispersion in the core of the double-clad optical fiber;

FIG. 2E is a block diagram illustrating one exemplary embodiment of a photodetector that comprises either a photomultiplier tube (PMT) or an avalanche photodiode (APD);

FIG. 2F is a block diagram illustrating an alternative photodetector that includes a spectrometer, followed by a charge coupled device (CCD) array;

FIGS. 3A and 3B respectively illustrate TPF images of 6-μm, and 2.2-μm fluorescence beads, wherein the blurriness of the 2.2-μm beads image indicates that the lateral resolution limit of the current endoscope is being reached (scale bars are 10 μm);

FIG. 4 illustrates a TPF image of breast cancer (SK-BR-3) cells targeted by fluorescein-labeled antibodies, which bind to cell surface proteins, where the excitation power in the core of the fiber is ˜10 mW; and

FIGS. 5A and 5B respectively illustrate images of a resolution chart and a straight edge, before and after correction for phase lag distortion is applied.

### DESCRIPTION

Figures and Disclosed Embodiments Are Not Limiting

Exemplary embodiments are illustrated in referenced Figures of the drawings. It is intended that the embodiments and Figures disclosed herein are to be considered illustrative rather than restrictive. No limitation on the scope of the technology and of the claims that follow is to be imputed to the examples shown in the drawings and discussed herein.

Optical Fiber Scanning Device

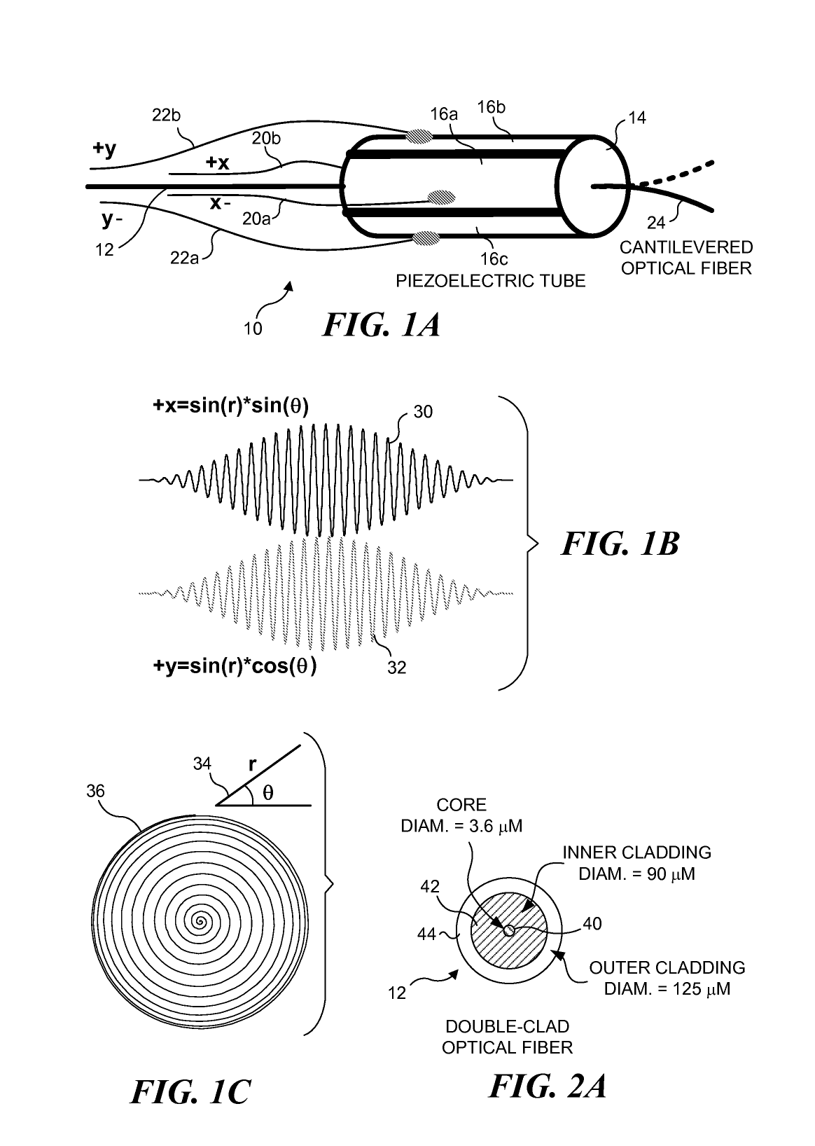

The scanning mechanism for the exemplary endoscope discussed below is an adaptation of a design initially developed for real-time optical coherence tomography. The endoscope in this exemplary embodiment includes a compact scanning device (shown in FIG. 1A) comprising a piezoelectric tube 10 and having a piezoelectric actuator 14 that drives a cantilevered optical fiber 24 to move in a desired scanning pattern. The outer surface of piezoelectric tube 10 is divided into four quadrants (of which only three quadrants 16a, 16b, and 16c are visible in this view), forming two pairs of drive electrodes. The drive signals applied to these electrodes through two pairs of electrical leads 20a and 20b, and 22a and 22b are modulated to drive cantilevered optical fiber 24 to move in the desired scanning pattern relative to two generally orthogonal axes, i.e., relative to ±x and ±y coordinates. A circular beam scanning pattern is obtained when appropriately modulated sine and cosine drive waveforms are applied to piezoelectric actuator 14 and a pulsed light produced by an external source (not shown in this Figure) is conveyed through a core of a double-clad optical fiber 12, which is coupled to (or comprises) cantilevered optical fiber 24, so that the pulsed light exits from the distal tip of the moving cantilevered optical fiber, to scan a target region, sample, or other region of interest. In this exemplary embodiment, drive signals at or near the mechanical resonant frequency of the cantilevered optical fiber are applied to the two pairs of electrodes, so that the cantilevered optical fiber vibrates at about its resonant frequency. It should be understood that by providing appropriate modulated drive signals the desired scanning pattern can comprise either: a helical (spiral scan), a linear scan, a raster scan, a circular scan, a Lissajous pattern scan, a rotating propeller scan, or any of a number of other types of space-filling scanning patterns.

By modulating the drive voltage with appropriate triangular or sinusoidal waveforms, a spiral scanning pattern is achieved in this exemplary embodiment. For example, for a probe with a free-standing cantilevered optical fiber length of ˜8.2 mm (with a diameter of about 125 μm), the scanning frequency ranges from about 1,323 to about 1,330 Hz for reasonable maximum scanning diameters of approximately 120-220 μm, using a maximum peak-to-peak drive voltage of about 75 volts. The overall diameter of the scanning endoscope is about 2.4 mm in this exemplary embodiment. However, it should be understood that none of the dimensions presented in this disclosure are intended to be in any way limiting on the scope of the concept. For example, endoscopes with longer or shorter cantilevered optical fibers can be employed, as well as endoscopes that have different diameters than this exemplary embodiment.

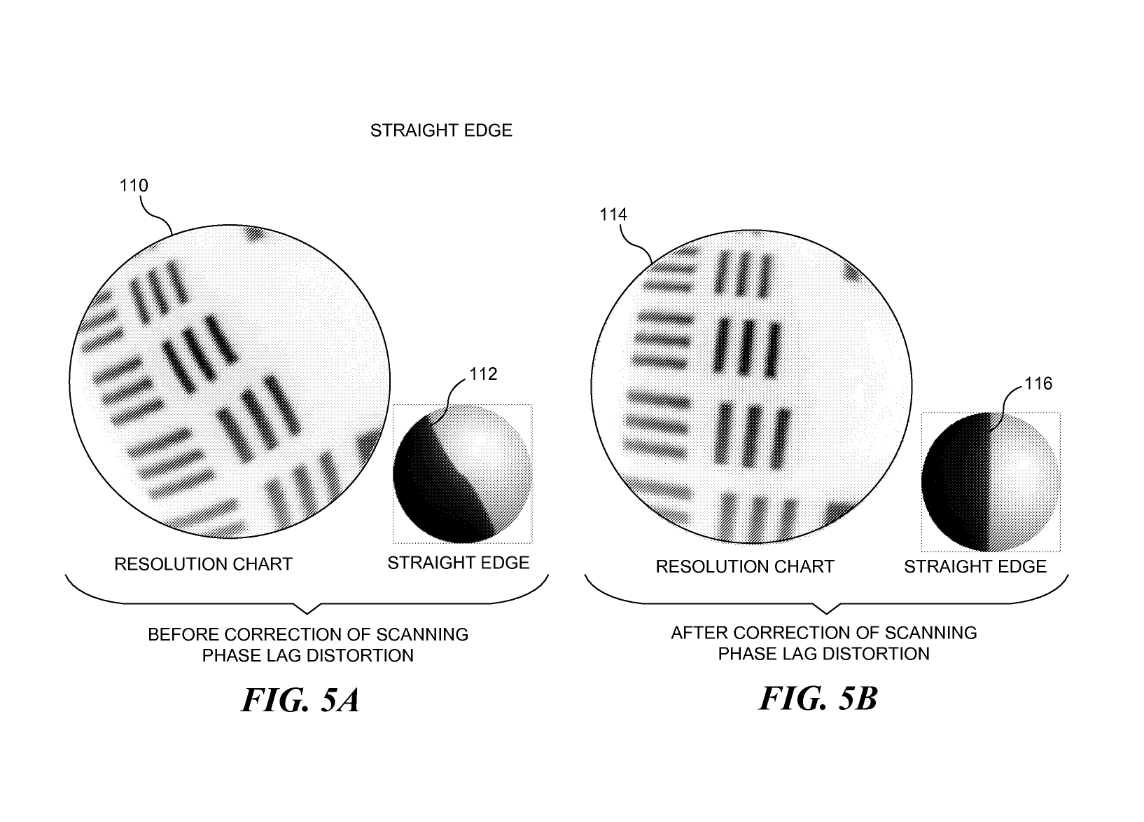

Modulating the sinusoidal drive waveform with a triangular envelope results in a spiral scanning pattern where the radius varies linearly in time. Unfortunately, discontinuity in the derivative of the modulation amplitude causes the probe to ring, distorting the image. Replacing the triangular modulation with smooth sinusoidal modulation envelopes 30 and 32 used to drive the piezoelectric actuators relative to the two orthogonal axes (see FIG. 1B) eliminates or at least reduces this artifact. The time-dependent sinusoidal function is then used to calculate the radius at which each sample is scanned. During spiral scanning to produce a spiral scanning pattern 36 as shown in FIG. 1C, the probe's angular response 34 will in general lag behind the drive waveform (applied on the ±x and ±y drive electrodes). The amount of lag depends on the amplitude of the modulation envelope (namely, the instantaneous radius of the spiral scan), causing objects to appear ‘twisted’ about the origin of the scanning pattern. An image 110 of a resolution chart shown in FIG. 5A illustrates this distortion. Correction of this image distortion is straightforward, given the estimate of the total angular lag as a function of radius. The image of a target of a fixed pattern (such as straight edge passing through the spiral scan center) will be curved in a simple way reflecting the angular lag and thus serves as a convenient calibration image, as also shown in an image 112 in FIG. 5A. It has been experimentally confirmed that the angular lag function is stable for a given spiral scanning frequency and the radial scanning direction (e.g., the “opening spiral” and the “closing spiral” images exhibit different lags). The results of applying a correction to images 110 and 112 are respectively illustrated in FIG. 5B, which illustrates an image 114 of the resolution chart and an image 116 of the straight edge, showing the results of correcting for the scanning phase lag distortion in this manner.

Double-Clad Optical Fiber

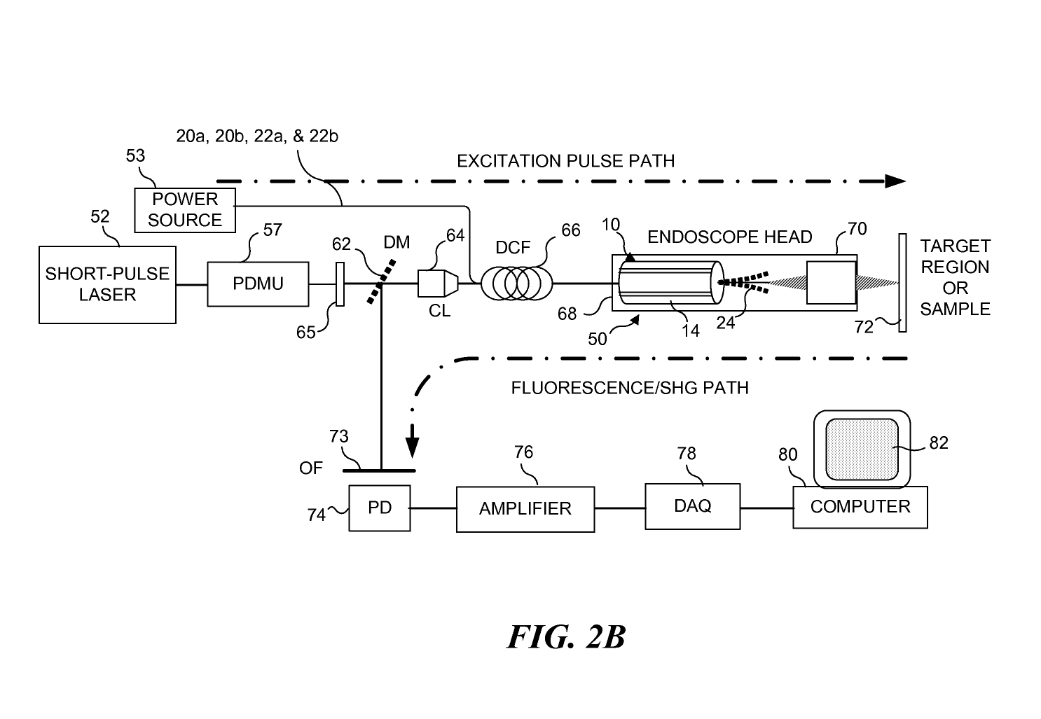

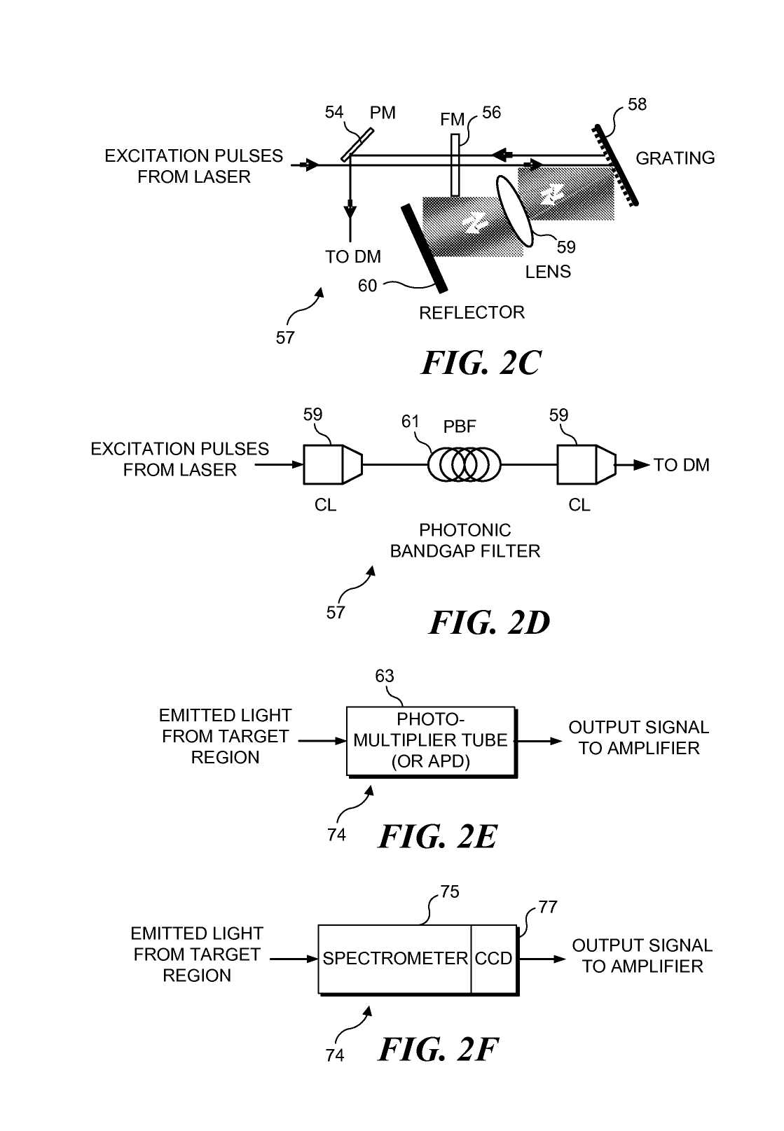

For light delivery to a sample or target region, and emitted light collection arising from excitation of molecules at the sample or target region, a commercially available double-clad optical fiber 12 (for example, available from Fibercore Ltd.) was used. Double-clad optical fibers are characterized by having a central single-mode core 40 surrounded by an inner cladding 42, and an outer cladding 44, as shown in the example of FIG. 2A, although it is clearly contemplated that additional claddings can be employed in multi-cladding optical fibers, which would also be useful in the present approach. The core and the inner cladding of the exemplary double-clad optical fiber have diameters of 3.6 μm and 90 μm, and numerical apertures (NA) of 0.19 and 0.23, respectively, although it will be understood that none of these values are intended to be limiting on the scope of this technology. A double-clad fiber with a larger inner cladding and higher NAs for the core and inner cladding can be used to improve emitted light collection efficiency. Previous research has demonstrated the use of double-clad optical fibers for enhancing fluorescence collection, where the same optical fiber is used for conveying excitation light (through the core), as well as for the collection of MPF (through the core and inner cladding) that is emitted by any fluorophore molecule disposed at the target region illuminated with the excitation light. The enhancement is attributed to the property of double-clad optical fibers that enables light to propagate in the inner cladding region by total internal reflection. Consequently, the collection area of the exemplary double-clad optical fiber is about 400 times larger compared to conventional single mode optical fibers. The NA of inner cladding 42 is also twice as large as the NA of a conventional single mode fiber. The large collection area and increased NA also make the collection efficiency less sensitive to chromatic aberrations of any lens used as an objective lens distally of the cantilevered optical fiber, such as a GRIN lens, or an achromatic micro-compound lens.

Exemplary System

An exemplary embodiment of a scanning optical fiber endoscope system 50 for nonlinear optical imaging and spectroscopy is shown in FIG. 2B. Short pulses (i.e., with pulse widths ranging from a few femtoseconds to tens of picoseconds) from a laser 52 pass through a pulse dispersion management unit (PDMU) 57, which compensates for pulse broadening caused by positive dispersion that occurs in the double-clad optical fiber core, as the pulsed light from the laser is conveyed toward the sample or target region.

One exemplary embodiment of pulse dispersion manager 57, which is illustrated in FIG. 2C, comprises a pulse stretcher that includes a beam pickoff mirror (PM) 54, a folding mirror (FM) 56, a grating 58, a lens 59, and a reflector 60. The excitation pulses from short-pulse laser 52 are incident on grating 58, which separates the light comprising the pulses based on wavelength. The differences in the lengths of the paths followed by the different wavelengths of the light from the grating before the light is reflected by reflector 60 are selected to compensate for the pulse broadening effect in the core of the double-clad optical fiber that conveys the pulsed light toward the sample or target region. Using folding mirror FM 56, the light within the stretcher experiences a double-pass, and is then reflected by PM 54 towards a dichroic mirror 62 (shown in FIG. 2B). In this exemplary embodiment for the pulse stretcher of PDMU 57, a 1200 lines/mm gold-coated reflection grating 58 is employed in a double-pass configuration. Optimal dispersion introduced in the pulse stretcher was determined by focusing the output of the endoscope into a BBO crystal (i.e., into a beta-BaB204 crystal, which was used for frequency doubling—not shown) and maximizing the second harmonic signal through the adjustment of the grating-to-lens separation (or by adjustment of an angle formed between the path of the incident pulsed light and a line that is normal to the surface of the grating).

A simpler exemplary embodiment for pulse dispersion management unit 57 is shown in FIG. 2D. In this embodiment, a photonic bandgap filter (PBF) 61 receives the excitation pulses from the short-pulse laser through a coupling lens (CL) 59. The pulsed light travels through the PBF, is re-collimated through another CL 59, and is then directed to DM 62 (as shown in FIG. 2B). The length of PBF 61 (of a given structure or configuration) determines the compensation that it provides for the pulse broadening effect that occurs in the core of double-clad optical fiber. Thus, when using a PBF for negatively pre-chirping the excitation pulsed light, the total negative dispersion is controlled by the length and other characteristics of a specific type of PBF employed.

Referring again to FIG. 2B, the pulsed light that has been compensated by pulse stretching is directed from PDMU 57 towards a dichroic mirror (DM) 62. DM 62 is coupled to a double-clad optical fiber 66 of an optical fiber endoscope 68 through a coupling lens (CL) 64. At the proximal end of the double-clad optical fiber, electrical leads 20a, 20b, 22a, and 22b are connected to an external power supply 53, which provides the required modulated drive signals to drive piezoelectric actuator 14 to move the cantilevered optical fiber in the desired scanning pattern. The optical fiber endoscope includes piezoelectric tube 10 with piezoelectric actuator 14, for moving cantilevered optical fiber 24 in a desired scanning pattern, as described above. Pulsed light traveling through double-clad optical fiber 66 exits from the distal end of cantilevered optical fiber 24, passes through a micro-lens 70 (such as a GRIN objective lens or alternatively, a micro achromatic compound lens, a micro-spherical lens, or an aspherical lens), and scans a sample or target region 72 with the pulsed light in the desired scanning pattern. Molecules at tissue in the target region or sample are excited by the pulsed light. Due to the absorption of two or more photons of the exciting pulsed light, the energy state of electrons in the fluorophore molecules is increased from the ground state to an elevated state. As the electrons of the fluorophore molecules decay back to their ground state, they produce emitted light, which comprises MPF. Depending upon the molecules that are present at the sample or target region, the emitted light can also comprise SHG light. The emitted light can be used for producing MPF images and/or SHG images, and can convey spectroscopic information that can be detected and imaged.

Thus, MPF comprises a fluorescence signal that is from the target region and is collected by micro-lens 70 (e.g., a GRIN lens with NA=0.46, a magnification of ˜0.7, and a working distance of ˜0.9 mm in this exemplary embodiment, although such details should not be considered to be in any way limiting). The fluorescence signal is conveyed through the core and inner cladding of cantilevered optical fiber 24 and double-clad optical fiber 66. (Note that the cantilevered optical fiber can comprise the distal end of the double-clad optical fiber or can be mechanically coupled to the distal end of the double-clad optical fiber, e.g., so that the core, inner cladding, and outer cladding portions of the double-clad optical fiber are thermally fused or mechanically or adhesively bonded to the corresponding component portions of the cantilevered optical fiber.) The fluorescence signal exiting the proximal end of double-clad optical fiber 66 is directed towards a photodetector (PD) 74 using DM 62, and residual excitation light is further blocked by an optical filter (OF) 73, which can comprise both a short-pass filter (e.g., with a cut-off wavelength of 650 nm) and a bandpass filter (e.g., passing light with wavelengths in the range from 350 nm-650 nm), which is disposed in front of the PD.

Finally, the signal from PD 74 is amplified by an amplifier 76, and digitized and conditioned by a data acquisition system (DAQ) 78. The conditioned digital signal is supplied to a computer 80 (or other computing device or processor) for processing and can either be stored and/or displayed on a display monitor 82, which enables MPF, and/or SHG, and/or spectroscopic images to be viewed and further analyzed by medical personnel. Theses images can be used for various purposes, such as to determine whether cancerous cells are present at the sample or target region.

Photodetector (PD)

As indicated in an exemplary embodiment of PD 74 illustrated in FIG. 2E, the PD can be a photomultiplier tube (PMT) 63, or alternatively, can comprise another type of light sensitive device, such as an avalanche photodiode (APD). Either the PMT or APD is suitable for PD 74 in producing MPF and SHG images. When used for SHG imaging, the system is basically the same as the system used for MPF imaging, except that for OF 73, the bandpass filter disposed in front of the PD 74 will be different. For SHG, the bandpass filter should generally have a much narrower bandwidth compared to the bandpass filter for MPF. The bandwidth of the bandpass filter used for SHG can be about equal to the quotient of the bandwidth of the excitation spectrum divided by \sqrt{2}, with the center bandwidth wavelength located at about the midpoint of the excitation spectrum peak wavelength. In addition, a half-wave plate (HWP) 65 can be used (e.g., by disposing it between PDMU 57 and the DM 62) for adjusting the polarization of the excitation pulses comprising the pulsed light, in order to maximize the SHG light produced in the target region.

For MPF spectroscopy, PD 74 can comprises a spectrometer 75, followed by a charge coupled device (CCD) array 77. Thus, imaging can be performed spectroscopically to produce MPF images at any desired specific wavelength within the MPF spectrum range. The scanning endoscope can also perform MPF spectroscopy imaging. For this type of application, spectrometer 75 is an imaging spectrometer, and CCD array 77 is used to read out spectroscopic information (i.e., the intensity at different wavelengths of the MPF emission spectrum). The spectroscopy (or wavelength dependent) information can facilitate the differentiation of abnormal tissue from normal tissue, which provides another avenue of disease detection in addition to the overall MPF intensity images.

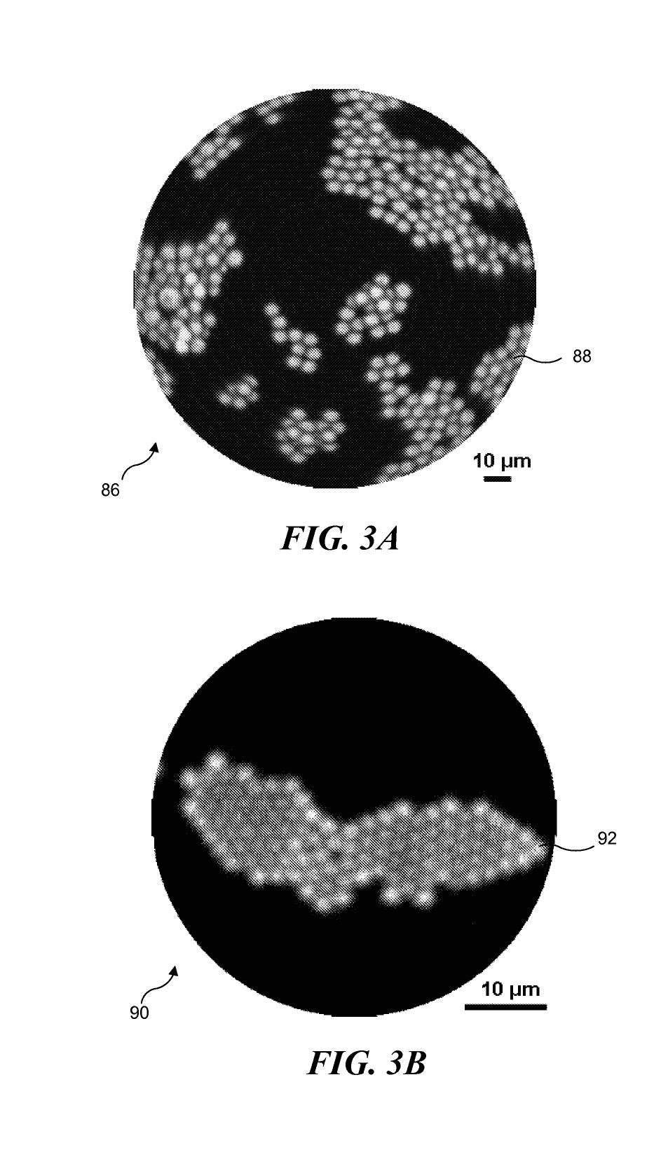

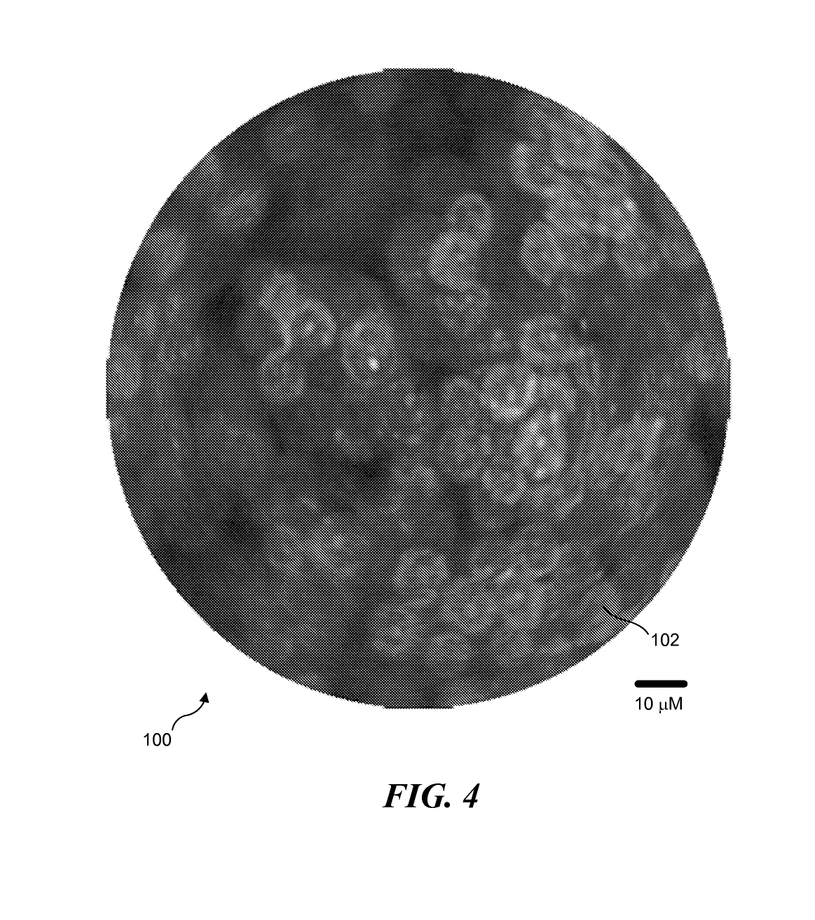

FIGS. 3A and 3B respectively show exemplary images 86 and 90 of 6-μm and 2.2-μm fluorescence beads 88 and 92. The frame rate of an exemplary circular image comprising 512 rings and 521 pixels per ring is approximately 2.6 Hz. The frame rate can be increased by constructing a probe with a shorter cantilevered optical fiber, since the resonance frequency (i.e., the spiral scanning frequency) of the cantilevered optical fiber is inversely proportional to the square of the cantilever length. Alternatively, depending on the spot size of the beam at the sample and the desired scanning range, the number of rings per image can also be reduced to increase the frame rate. The parameters used in this example result in over-sampling. The excitation power delivered to the sample is about 10 mW (through the core of the optical fiber). In addition to fluorescence beads, imaging of fixed breast cancer cells (SK-BR-3) 102 targeted by fluorescein labeled antibodies has been performed, as illustrated in a MPF image 100 in FIG. 4, indicating potential applications of the scanning optical fiber endoscope for imaging biological samples. Note that this exemplary scanning endoscope, which uses the same cantilevered optical fiber for excitation pulse delivery and for multiphoton fluorescence collection, can also be conveniently used externally for imaging tissue on the surface of the body or tissue samples that have been collected from a patient or non-biological fluorescent or SHG samples.

The image of 2.2-μm fluorescence beads 92, which is shown in FIG. 3B, illustrates that the lateral resolution limit of the current probe is being reached (because the image of the beads is slightly blurred). A Gaussian fit to the fluorescence intensity as a 0.5-μm fluorescence bead is scanned across the beam focus gives a lateral point-spread function width of 2.0±0.2 μm (i.e., the full-width-at-half-maximum), which is close to the 2.5 μm focused spot size that is predicted. The axial resolution is measured by recording the multiphoton fluorescence signal level as the probe is axially scanned through a layer of 0.5-μm fluorescence beads. A Gaussian fit to the signal gives an axial point spread function width of about 20 μm. The resolution parameters can be further improved with improved design of the lens assembly. Optical fibers with larger NAs that will fully utilize the NA of the GRIN lens can further increase the detected signal levels.

In the exemplary system discussed above, only the linear dispersion of the double-clad optical fiber is compensated. Previous studies have shown that even at moderate pulse energies, self-phase modulation can significantly modify pulse widths and affect the generation of multiphoton fluorescence. In this exemplary system, the typical average power in the core of the fiber is 10 mW. Previous research has shown that in this range of power levels, nonlinear pulse propagation can begin to modify the pulse shape and spectrum, which in turn, can reduce the multi-photon excitation efficiency. The use of special optical fibers with low dispersion values or the implementation of appropriate schemes to reduce the nonlinear dispersion effects is expected to improve the signal levels.

Thus, an exemplary optical fiber endoscope for scanning MPF, SHG, in real-time imaging, and collecting spectroscopic information has been developed, as discussed above. A piezoelectric actuator for optical fiber tip scanning enables real-time imaging in vivo, and a double-clad optical fiber that is used for both excitation light delivery and collection of the MPF light addresses some of the key challenges associated with the use of a conventional single mode optical fiber (i.e., endoscopic beam scanning and low collection efficiency). By using the same optical fiber for both delivery of the excitation pulsed light and collection of emitted light from molecules at the site, a flexible and compact endoscope has been constructed, which can be potentially integrated with existing endoscopic technology for real-time, in vivo imaging of internal organs, and for other applications, as will be evident to one of ordinary skill in the art.

Although the concepts disclosed herein have been described in connection with the preferred form of practicing them and modifications thereto, those of ordinary skill in the art will understand that many other modifications can be made thereto within the scope of the claims that follow. Accordingly, it is not intended that the scope of these concepts in any way be limited by the above description, but instead be determined entirely by reference to the claims that follow.

The invention in which an exclusive right is claimed is defined by the following:

1. A system for capturing light emissions from a target region within a patient's body, comprising:
(a) a light source that produces a pulsed light;
(b) an optical fiber having a core covered by a plurality of claddings, a proximal end, and a distal end, the core being configured to couple at the proximal end of the optical fiber to the light source producing the pulsed light, for conveying the pulsed light to the distal end of the optical fiber through the core;
(c) a cantilevered optical fiber that includes a core within a plurality of claddings, the cantilevered optical fiber being coupled to the distal end of the optical fiber to receive the pulsed light that is conveyed through the optical fiber, so that the pulsed light is conveyed through the core of the cantilevered optical fiber and exits from a free end of the cantilevered optical fiber;
(d) an actuator for driving the cantilevered optical fiber to move, so that the pulsed light exiting from the free end scans in a desired scanning pattern;
(e) a lens to focus the pulsed light traveling from the free end of the cantilevered optical fiber toward a target region within a patient's body, so that the pulsed light excites molecules at the target region to emit light in response to the pulsed light, and to focus emitted light received from the target region back into the core and an inner cladding of the cantilevered optical fiber, the emitted light being conveyed through the cantilevered optical fiber and through the core and an inner cladding of the optical fiber that is coupled thereto toward the proximal end of the optical fiber;
(f) a splitter that separates the emitted light conveyed back through the optical fiber from the pulsed light that is produced by the light source, so that the emitted light exiting the proximal end of the optical fiber is conveyed along a detection path, while the pulsed light is introduced into the proximal end of the optical fiber and conveyed thereby to the target region;
(g) a photodetector disposed in the detection path, for responding to the emitted light and producing a corresponding electrical output signal; and
(h) a processor for processing the electrical output signal, for use in determining a characteristic of the target region based upon the emitted light.

2. The system of claim 1, wherein the photodetector comprises an imaging photodetector and wherein the output signal comprises an image signal that is processed to produce an image of the target region in response to the emitted light.

3. The system of claim 2, wherein the emitted light comprises multiphoton fluorescence (MPF) that is emitted by molecules at the target region that have each absorbed multiple photons of the pulsed light, the electrical output signal being used to produce an MPF image of the target region.

4. The system of claim 1, wherein the photodetector comprises a spectrometer disposed in front of an imaging device that produces the output signal, so that the output signal is responsive to a spectral content of the emitted light from the target region.

5. The system of claim 4, wherein the emitted light comprises multiphoton fluorescence (MPF), and wherein the electrical output signal produced by the photodetector comprising the spectrometer and CCD is processed to produce MPF images at a desired specific wavelength within a spectrum range of the MPF.

6. The system of claim 4, wherein the emitted light comprises multiphoton fluorescence (MPF), and wherein the spectrometer is an imaging spectrometer and the electrical output signal is processed to produce spectroscopic information indicative of an intensity of different wavelengths of an MPF emission spectrum.

7. The system of claim 1, wherein the emitted light is generated as a second harmonic of the pulsed light, due to the absorption of two photons of the pulsed light by molecules at the target region, which then produce emitted light that is at twice an energy level of each of two photons comprising the pulsed light absorbed by non-centrosymmetric molecules at the target region, the emitted light thus comprising second harmonic generation (SHG) light.

8. The system of claim 7, further comprising a polarization controller for adjusting a polarization of the emitted light, in order to substantially maximize the SHG light produced in the target region.

9. The system of claim 1, wherein the splitter comprises a dichroic mirror that transmits light of a first waveband, while reflecting light of a second waveband that is substantially different than the first waveband, the pulsed light having a waveband that is substantially equal to one of the first and the second wavebands, and the emitted light having a waveband that is substantially equal to the other of the first and second wavebands.

10. The system of claim 1, wherein the actuator drives the cantilevered optical fiber to move in the desired scanning pattern defined relative to two generally orthogonal axes.

11. The system of claim 10, wherein the actuator is energized by a drive signal modulated with a voltage waveform selected from a group consisting of a triangular waveform; a sinusoidal waveform; and modified versions of the triangular and sinusoidal waveforms.

12. The system of claim 1, wherein the desired scanning pattern comprises a pattern selected from the group consisting of:
(a) a linear scan pattern;
(b) a raster scan pattern;
(c) a spiral scan pattern;
(d) a propeller scan pattern;
(e) a Lissajous scan pattern; and
(f) a circular scan pattern.

13. The system of claim 1, further comprising a pulse dispersion manager disposed in a path between the light source and the proximal end of the optical fiber, the pulse dispersion manager negatively pre-chirping pulses of the pulsed light to compensate for a pulse broadening caused by a positive dispersion of the pulsed light within the core of the optical fiber having the plurality of claddings.

14. The system of claim 13, wherein the pulse dispersion manager comprises a pulse stretcher that includes a grating, a lens, and a plurality of reflective surfaces.

15. The system of claim 13, wherein the pulse dispersion manager comprises a photonic bandgap filter selected to have a desired negative dispersion to compensate for the pulse broadening in the core of the optical fiber having the plurality of claddings.

16. The system of claim 1, further comprising a coupling lens for coupling the pulsed light into the core at the proximal end of the optical fiber.

17. The system of claim 1, wherein the lens that focuses pulsed light exiting from the free end of the cantilevered optical fiber comprises a micro-lens selected from the group consisting of:
(a) a gradient index (GRIN) lens;
(b) a micro achromatic compound lens;
(c) a micro-spherical lens; and
(d) an aspherical lens.

18. The system of claim 1, wherein the light source that produces the pulsed light comprises a laser that produces the pulsed light with a pulse width on the order of from several femtoseconds to several tens of picoseconds.

19. The system of claim 1, wherein the actuator comprises a tubular piezoelectric actuator.

20. A scope for use in producing and collecting light emissions from a target region within a patient's body, comprising:
(a) an optical fiber having a core covered by a plurality of claddings, a proximal end, and a distal end, the core being configured to couple at the proximal end of the optical fiber to a light source producing a pulsed light, for conveying the pulsed light to the distal end of the optical fiber through the core;
(b) an elongate housing, which is disposed at the distal end of the optical fiber;
(c) a scanning device that is disposed within the housing and is coupled to the distal end of the optical fiber to receive the pulsed light conveyed through the core of the optical fiber, the scanning device having a free end that is actuated to scan in a desired scanning pattern, so that the pulsed light exiting from the free end scans a target region within a patient's body; and
(d) a lens disposed within the housing and configured for focusing the pulsed light exiting from the free end of the scanning device onto a target region and for focusing emitted light from the target region back into the scanning device, the emitted light being conveyed through the core and an inner cladding of the optical fiber to the proximal end of the optical fiber, which is configured to couple the emitted light onto a photodetector.

21. The scope of claim 20, wherein the scanning device includes an actuator, and a cantilevered optical fiber, the cantilevered optical fiber having a proximal end coupled to the optical cable and driven to move by the actuator, and a distal free end through which the pulsed light exits and through which the emitted light is received, the actuator being configured to connect via electrical leads that extend proximally from the actuator to an external power source that can energize the actuator to drive the cantilevered optical fiber to move in the desired scanning pattern.

22. The scope of claim 21, wherein the actuator comprises a piezoelectric actuator that is able to drive the cantilevered optical fiber to move relative to at least one axis that is transverse to a longitudinal axis of the actuator.

23. The scope of claim 21, wherein the cantilevered optical fiber includes a core around which is disposed a plurality of cladding layers, the pulsed light being conveyed through the core of the cantilevered optical fiber, and the emitted light being received at the free end of the cantilevered optical fiber and conveyed through the core and an inner cladding of the cantilevered optical fiber and into the core and the internal cladding of the optical fiber.

24. The scope of claim 20, wherein the lens comprises a micro-lens selected from the group consisting of:
(a) a gradient index (GRIN) lens;
(b) a micro achromatic compound lens;
(c) a micro-spherical lens; and
(d) an aspherical lens.

25. A method for producing light emissions from a target region in a patient's body, comprising the steps of:
(a) introducing pulsed light into a proximal end of an optical fiber having a core and a plurality of cladding layers, the pulsed light being conveyed by the core to a distal end of the optical fiber that is configured to be advanced to a position proximate to the target region;
(b) actuating a scanning device disposed at the distal end of the optical fiber where the scanning device receives the pulsed light, movement of the scanning device causing the pulsed light to scan the target region in a desired scanning pattern;
(c) focusing the pulsed light from the scanning device onto the target region;
(d) focusing emitted light received from the target region into the scanning device, the emitted light being produced in response to excitation of molecules at the target region by the pulsed light;
(e) conveying the emitted light received from the target region back through the core and an inner cladding layer of the optical fiber, to the proximal end of the optical fiber;
(f) directing the emitted light exiting the optical fiber so that the emitted light is incident on a photodetector, the photodetector producing a signal indicative of a characteristic of the target region; and
(g) producing an image responsive to the signal output by the photodetector for evaluating a condition of the target region.

26. The method of claim 25, wherein the scanning device moves a cantilevered optical fiber relative to two axes, to scan the target region in the desired scanning pattern, the step of actuating the scanning device comprising the step of energizing the scanning device with modulated signals to move the cantilevered optical fiber relative to the two axes.

27. The method of claim 25, wherein the desired scanning pattern comprises a space-filling pattern.

28. The method of claim 25, further comprising the step of negatively pre-chirping pulses of the pulsed light before the step of introducing the pulsed light into the optical fiber, to compensate for a pulse broadening caused by a dispersion of the pulsed light within the core of the optical fiber.

29. The method of claim 25, wherein the steps of focusing the pulsed light exiting from the scanning device onto the target region comprises the steps of passing the pulsed light through a micro-lens.

30. The method of claim 29, wherein the steps of focusing the emitted light received from the target region into the scanning device comprises the step of passing the multiphoton light through the micro-lens.

31. The method of claim 25, wherein the emitted light comprises multiphoton fluorescence (MPF) light that is emitted by molecules that have each absorbed multiple photons of the pulsed light, and wherein the step of producing the image comprises the step of producing an MPF image of the target region in response to the MPF light emitted by the molecules at the target region.

32. The method of claim 31, wherein the step of producing the image further comprises the step of detecting the MPF light at a desired specific wavelength within an MPF spectrum range.

33. The method of claim 31, wherein the step of producing the image further comprises the step of producing MPF spectroscopic images by detecting an intensity of the MPF light at different wavelengths of an MPF emission spectrum.

34. The method of claim 25, wherein the emitted light is generated as a second harmonic of the pulsed light, due to the absorption of two photons of the pulsed light by molecules at the target region, which then produce emitted light that is at twice an energy level of each of two photons comprising the pulsed light that is absorbed by non-centrosymmetric molecules at the target region, the emitted light thus comprising second harmonic generation (SHG) light, wherein the step of producing the images comprises the step of producing SHG images of the target region in response to the SHG light.

35. The method of claim 34, further comprising the step of adjusting a polarization of the pulsed light, to substantially maximize the SHG light produced in the target region.
